(19) United States
(12) Patent Application Publication
(10) Pub. No.: US 2022/0309656 A1
(43) Pub. Date: Sep. 29, 2022

(54) OPHTHALMOLOGIC IMAGE PROCESSING METHOD AND OPHTHALMOLOGIC IMAGE PROCESSING DEVICE

(71) Applicant: NIDEK CO., LTD., Gamagori (JP)

(72) Inventors: Tetsuya KANO, Gamagori (JP); Ryosuke SHIBA, Gamagori (JP); Haruka UEMURA, Gamagori (JP)

(73) Assignee: NIDEK CO., LTD., Gamagori (JP)

(21) Appl. No.: 17/671,934

(22) Filed: Feb. 15, 2022

(30) Foreign Application Priority Data
Mar. 29, 2021 (JP) 2021-056271

(51) Int. Cl. G06T 7/00; G06T 3/40; G06T 7/30; A61B 3/10; A61B 3/00

(52) U.S. Cl. CPC G06T 7/0012; G06T 3/40; G06T 7/30; A61B 3/102; A61B 3/0041; G06T 2207/30041; G06T 2207/10101

(57) ABSTRACT

A processor of an ophthalmologic image processing device acquires a plurality of B scan OCT data of a tissue of a subject eye, in which the plurality of B scan OCT data is captured at different scan positions, collectively sets an extraction region in the plurality of B scan OCT data to include an image of the subject eye in each of the plurality of B scan OCT data, and displays, on a predetermined display region, a plurality of extracted OCT data extracted from the respective B scan OCT data. Each of the plurality of extracted OCT data is switched to be alternatively displayed.

### CROSS-REFERENCE TO RELATED APPLICATION

This application claims priority from Japanese Patent Application No. 2021-056271 filed on Mar. 29, 2021, the entire subject-matter of which is incorporated herein by reference.

### TECHNICAL FIELD

The present disclosure relates to an ophthalmologic image processing method and an ophthalmologic image processing device for processing OCT data of a tissue of a subject eye.

### BACKGROUND ART

In recent years, in the field of ophthalmology, an optical coherence tomography (OCT), which is a device for capturing a tomographic image of a tissue of a subject eye, has attracted attention.

In an OCT device of the related art, since an effective imaging range in a depth direction called a depth range is narrow, an entire imaging range is displayed as it is in a case of displaying captured OCT data (refer to, for example, JP-A-2016-055122).

In an examination performed on expanding of the depth range, even in a case where the depth range was expanded, an actually necessary depth-direction imaging range was different for each subject eye. For example, in a case of capturing an image of a fundus, there are individual differences in a height difference of a fundus tissue due to an axial length and a curvature of the fundus, and thus it is considered that the actually necessary depth-direction imaging range is different for each subject eye.

Further, as the depth range is widened, a proportion of an image of the subject eye in the OCT data is relatively small, and a proportion of the margin is large. Therefore, as in JP-A-2016-055122, in a case where the entire imaging range is to be displayed as it is, it is considered that it is difficult to observe the tissue as the display range on the screen becomes enlarged or the image of the subject eye occupies a small proportion.

### SUMMARY OF INVENTION

An object of the present disclosure is to provide an ophthalmologic image processing method and an ophthalmologic image processing device capable of satisfactorily observing a desired tissue from OCT data having an extended depth range.

An ophthalmologic image processing method performed in an ophthalmologic image processing device, the ophthalmologic image processing method including:

an acquisition step of acquiring a plurality of B scan OCT data of a tissue of a subject eye, the plurality of B scan OCT data being captured at different scan positions;

a setting step of collectively setting an extraction region in the plurality of B scan OCT data to include an image of the subject eye in each of the plurality of B scan OCT data; and

a display control step of displaying, on a predetermined display region, a plurality of extracted OCT data extracted from the respective B scan OCT data, each of the plurality of extracted OCT data being switched to be alternatively displayed.

An ophthalmologic image processing device, including a processor configured to:

acquire a plurality of B scan OCT data of a tissue of a subject eye, the plurality of B scan OCT data being captured at different scan positions;

collectively set an extraction region in the plurality of B scan OCT data to include an image of the subject eye in each of the plurality of B scan OCT data; and

display, on a predetermined display region, a plurality of extracted OCT data extracted from the respective B scan OCT data, each of the plurality of extracted OCT data being switched to be alternatively displayed.

According to the present disclosure, it is easy to satisfactorily observe a desired tissue from OCT data having the extended depth range.

### BRIEF DESCRIPTION OF DRAWINGS

FIG. 1 is a diagram showing a schematic configuration of an OCT system according to an example.

FIG. 2 is a diagram showing an OCT optical system according to the example.

FIG. 3 is a flowchart showing an operation.

FIG. 4 is a diagram showing OCT data acquired on different scan lines.

FIG. 5A is a diagram showing OCT data acquired at a scan density of 1024 pt/9 mm.

FIG. 5B is a diagram showing OCT data acquired at a scan density of 512 pt/9 mm.

FIG. 6A is a diagram showing a state in which the entire OCT data is displayed on a display region.

FIG. 6B is a diagram showing a state in which extracted OCT data is displayed on the display region.

FIG. 7A is a diagram showing a scan pattern of a radial scanning

FIG. 7B is a diagram showing a scan pattern of a cross scanning

FIG. 7C is a diagram showing a scan pattern of a multi-cross scanning

### DESCRIPTION OF EMBODIMENTS

### Overview

An embodiment of the present disclosure will be described. The items classified by <> below can be used independently or in relation to each other.

### Overall Configuration

Hereinafter, one embodiment of the present disclosure will be described. First, an OCT system according to the embodiment will be described. The OCT system is used to capture an image of the subject eye and display an OCT data which is the result of the image capturing.

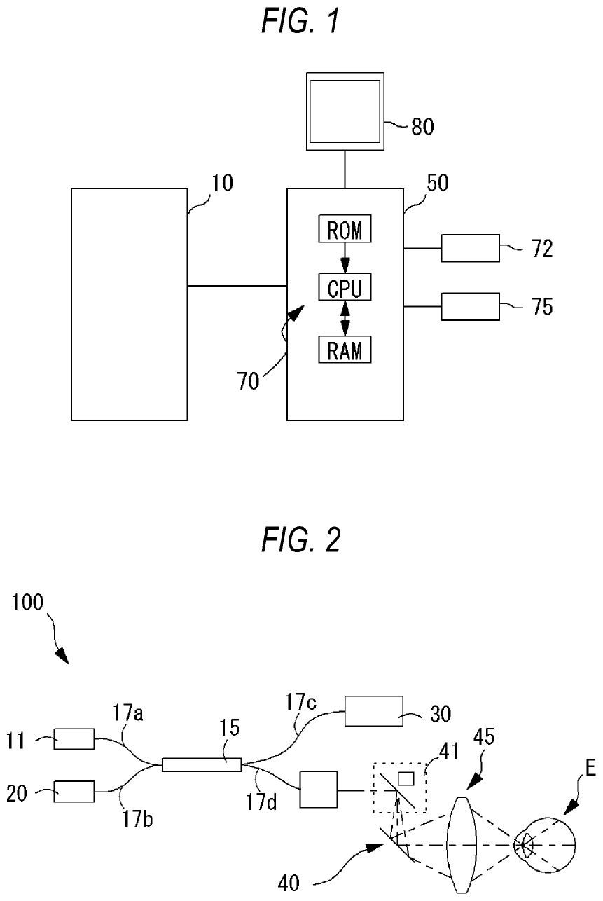

In the present embodiment, refer to FIG. 1, the OCT system includes at least an OCT optical system, an image processor, and an ophthalmologic image processing device. An ophthalmologic image processing program according to the embodiment is stored in a non-volatile memory that can be read by a processor of the ophthalmologic image processing device. The OCT optical system, the image processor, and the ophthalmologic image processing device may be integrated as an OCT device (for example, refer to examples). A part of the OCT optical system, the image processor, and the ophthalmologic image processing device may be separate from the other parts.

### OCT Optical System

The OCT optical system (refer to FIG. 2) is used to capture the OCT data of the subject eye. The OCT optical system detects a spectral interference signal of measurement light and reference light guided onto a tissue of the subject eye.

The OCT optical system may be a spectral domain OCT (SD-OCT) optical system or a swept source OCT (SS-OCT) optical system, for example. The OCT optical system is preferably suitable for acquiring the OCT data having a wide depth range. An OCT light source included in the SS-OCT optical system may be a vertical cavity surface emitting laser (VCSEL) type wavelength swept light source.

The OCT optical system may include at least one of a light splitting unit, a scanning unit (also referred to as an optical scanner), or a detector. The light splitting unit splits the light from the OCT light source into the measurement light and the reference light. The scanning unit is a device that scans the measurement light on the tissue of the subject eye. The scanning unit may be, for example, a combination of two optical scanners which has different scanning directions. The detector outputs a spectral interference signal by receiving the reference light and the measurement light guided to the subject eye. The OCT optical system may scan the measurement light along a plurality of predetermined scan lines on the tissue of the subject eye, and capture the OCT data of each of the plurality of scan lines. The scan line may be set at any position on the basis of an instruction from an examiner. Further, a scan line corresponding to a scan pattern may be set by selecting any of a plurality of predetermined scan patterns. Various scan patterns, such as line, cross, multi, map, radial, and circle, are known.

### Image Processor

The image processor performs processing on the spectral interference signal which is output from the OCT optical system to acquire the OCT data of the subject eye. The image processor may be shared by a control unit that controls an operation of the entire device in the OCT device, or may be an image processing device separate from the control unit.

### OCT Data

The OCT data may be signal data or visualized image data. For example, the OCT data may be at least one of tomographic image data representing reflection intensity characteristics of the subject eye, OCT angio data of the subject eye (for example, OCT motion contrast data), Doppler OCT data representing Doppler characteristics of the subject eye, or polarization characteristic data representing polarization characteristics of the subject eye.

The OCT data may be at least one of B scan data (for example, B scan tomographic image data, two-dimensional OCT angio data, and the like), front (En face) data (for example, OCT front data, front motion contrast data, and the like), or three-dimensional data (for example, three-dimensional tomographic image data, three-dimensional OCT angio data, and the like).

### Ophthalmologic Image Processing Device

The ophthalmologic image processing device acquires the OCT data of the subject eye generated by the image processor and displays the OCT data on, for example, a monitor. The OCT data may be displayed in an electronic report or a paper report. The ophthalmologic image processing device includes at least a processor (arithmetic control unit). The arithmetic control unit may include a CPU, RAM, ROM, and the like. In a case where the processor executes an ophthalmologic image observation program, the ophthalmologic image processing device performs each step described later. The ophthalmologic image observation program may be stored in a non-volatile storage medium accessible from the arithmetic control unit.

### Acquisition Step

First, an image of the subject eye is captured through the OCT optical system. As a result of capturing, the image processor generates the OCT data of the subject eye. After that, the OCT data which is generated by the image processor is acquired through the ophthalmologic image processing device. In the present embodiment, the plurality of B scan OCT data of the tissue of the subject eye captured at different scan positions are acquired. In this case, the plurality of B scan OCT data may be captured at different scan positions in an identical scan pattern.

### Setting Step

Next, in the setting step, an extraction region is set in the plurality of B scan OCT data. In the present embodiment, the extraction region of the plurality of B scan OCT data is set by the ophthalmologic image processing device. The extraction region is set to include an image of the subject eye in each of the plurality of B scan OCT data.

The extraction region may be set such that both a B scan OCT data in which a position of the upper end of the image of the subject eye is at the shallowest position and a B scan OCT data in which a position of the lower end of the image of the subject eye is at the deepest position, among the plurality of B scan OCT data, can be entirely viewed in the depth direction.

In a case of a fundus OCT, the position of the upper end of the image of the subject eye may be, for example, the upper end on a surface layer of a retina. Alternatively, it may be the upper end of a corpus vitreum on a boundary surface of the retinal side. The position of the lower end of the image of the subject eye may be, for example, the lower end of a choroid layer.

The upper and lower ends of the image of the subject eye may be determined by performing image processing on the OCT data. For example, the upper and lower ends of the image of the subject eye may be determined on the basis of the detection result of a layer boundary. Further, the upper and lower ends of the image of the subject eye may be determined on the basis of luminance information in the depth direction of the B scan OCT data.

In a case where the upper end and the lower end are obtained by image processing, at least one of the upper end or the lower end may be set with a predetermined margin with respect to the position of the tissue detected from the OCT data. According to this, the extraction region can be set to include a tissue (corpus vitreum, deep layer of the choroid layer, and the like) for which it is difficult to be detected through image processing.

### Change in Transverse Direction Range, in which Extraction Region Is Set

In the present embodiment, a transverse direction range in which the extraction region is set in the plurality of B scan OCT data may be changeable. In such a case, the extraction region is collectively set in the selected transverse direction range of each of the plurality of B scan OCT data.

The transverse direction range in which the extraction region is set in the plurality of B scan OCT data may be selectable from a plurality of transverse direction ranges different from each other. The selectable range may be provided in advance. For example, in the case of the fundus OCT data, the selectable range may be selected from at least a first range which is the central portion of the fundus (around the central fovea), and a second range which includes the central portion of the fundus and the peripheral part with respect to the central portion of the fundus.

By expanding the depth range, it is possible to capture a region having a larger height difference through one B scanning Hence, the imaging range in the transverse direction of the B scan OCT data can also be made longer (wider) than before. In observation and diagnosis using the fundus OCT, information about the central portion of the fundus is more important than information about the peripheral part. Since the transverse direction range in which the extraction region is set in the plurality of B scan OCT data can be changed, even in a case where the imaging range in the transverse direction is lengthened, the important part in diagnosis and observation is appropriately displayed as the extracted OCT data.

### Correction Step

A correction step may be performed to correct the mutual positional relationship of the plurality of B scan OCT data. A setting step may be performed after the correction step. That is, after the mutual positional relationship of the plurality of B scan OCT data is corrected, the extraction region may be set in each corrected B scan OCT data. In the correction step, the mutual positional relationship between the B scan OCT data may be corrected such that the same features are disposed at the same depth. For example, in each B scan OCT data, the mutual positional relationship may be corrected such that predetermined feature portions are detected and the positions of the detected feature portions (positions in the transverse direction and the depth direction) are aligned. The feature portions may be any of macula, papillae, blood vessels, and specific layers (or layer boundaries). In such a case, the extraction region is set in each B scan OCT data such that the positional relationship between the feature portion and the extraction region in each B scan OCT data matches each other.

In the correction step, the mutual positional relationship between the B scan OCT data may be corrected in consideration of curvature of the tissue in a direction intersecting a traverse section in which the B scan OCT data is acquired. In such a case, the mutual positional relationship may be corrected such that the image of the tissue in each B scan OCT data is disposed at the depth position where the curvature is reflected. In such a case, in each B scan OCT data, the extraction region is set at a depth position corresponding to each other in consideration of curvature.

In a case of correcting the mutual positional relationship, for example, affine transformation (parallel translation, shear correction, rotation correction) may be performed.

In addition, the image of the subject eye in the B scan OCT data may be inclined (tilted) due to the image being captured in a state of deviation in alignment, timing of improper fixation, and the like. Since the height difference of the image of the subject eye is enlarged by the tilting, the enlarged display of the image of the subject eye is likely to be limited in the display region. On the other hand, in the present embodiment, tilting correction may be performed on each B scan OCT data. For example, the tilt of the tissue of the subject eye with respect to the optical axis may be estimated on the basis of the alignment state at the time of imaging, and the B scan OCT data may be corrected in accordance with the estimated amount of tilt. As a result, the extraction region can be set more appropriately.

### Display Control Step

In a display control step, the plurality of extracted OCT data are displayed in a predetermined display region. The size of the display region is predetermined at least in the display medium. The arrangement on the display medium may be predetermined. The plurality of extracted OCT data are extracted from the extraction region set in each B scan OCT data. Each of the plurality of extracted OCT data is switched to be alternatively displayed in the display region. In the display region, an aspect ratio or the number of pixels in the vertical and horizontal directions (the number of device pixels) is predetermined. In the present embodiment, even in a case where the B scan OCT data having a wide depth range is acquired, without displaying the entire B scan OCT data, the depth region including the image position of the tissue is extracted from the B scan OCT data and displayed. Since the image of the tissue of the subject eye is displayed in a larger size with respect to the predetermined display region, the state of the tissue of the subject eye can be easily grasped through the extracted OCT data. In such a case, in the present embodiment, the region (extracted OCT data) extracted from the plurality of B scan OCT data and displayed in the display region is set to include the image of the subject eye in each of the plurality of B scan OCT data. Therefore, in a case where each of the plurality of extracted OCT data is switched to be alternatively displayed in a predetermined display region, the image of the subject eye is appropriately displayed. That is, regardless of the individual difference in the shape of the subject eye, the image of the subject eye is prevent from not being fit (not entirely viewed) in the display region in the depth direction.

In the correction step, the mutual positional relationship of the plurality of B scan OCT data is corrected. Therefore, the image of the subject eye can be appropriately enlarged and displayed in the display region.

### Magnification Adjustment

In the display control step, a lateral magnification of the extracted OCT data on the display region may be maintained constant among the B scan OCT data having different scan densities. In such a case, the lateral magnification may be adjusted, in accordance with the scan density of the B scan OCT data which is the basis of the extracted OCT data, to be constant.

### Display Setting for Each Scan Pattern

In a case where the extracted OCT data is displayed using the plurality of B scan OCT data captured at different scan positions in the identical scan pattern, it may be predetermined, for each type of the scan pattern, whether the extraction region is collectively set in the B scan OCT data captured at each scan position together with another B scan OCT data. For example, in a case where the extracted OCT data is displayed on the basis of the B scan OCT data in which the scanning directions match each other as in the one-way multi-scanning, the extraction region may be collectively set in the B scan OCT data captured at each scan position. On the other hand, for OCT data of which the scanning directions are orthogonal to each other, such as cross scanning, there may be a case where it is not necessary to match the extraction regions. Therefore, in such a case, the extraction region for each B scan OCT data may be set individually on the basis of the position of the image in each OCT data.

### Delete Information Other Than Extraction Region, and Save Data in Extraction Region

In the present embodiment, by setting the extraction region, the data in the region other than the extraction region may be reduced (deleted or compressed). As a result, the data in the extraction region (that is, the extracted OCT data) may be saved in the memory. The extracted OCT data may be saved alternatively with the original OCT data (for example, may be overwritten on the original OCT data). Even in a case where the depth range increases, the amount of OCT data to be saved can be reduced.

### EXAMPLE

Hereinafter, an OCT system (optical coherence tomography system) shown in FIGS. 1 and 2 will be described as an example.

As shown in FIG. 1, the OCT system according to the example includes at least an optical unit 10 and a control unit 50 corresponding to an ophthalmologic image processing device of the present example. In the present example, the optical unit 10 and the control unit 50 are integrated as an OCT device.

The optical unit 10 includes an OCT optical system 100 (refer to FIG. 2). Further, the control unit 50 is the ophthalmologic image processing device according to the present example, and includes at least an arithmetic control unit (processor) 70 that controls the entire OCT system. The arithmetic control unit (hereinafter, simply referred to as a control unit) 70 includes, for example, a CPU and a memory. As an example, in the present example, the control unit 70 also serves as an image processor in the OCT system.

The OCT system may be provided with a storage unit (memory) 72, an input interface (operation unit) 75, a monitor 80, and the like. Each unit is connected to the control unit 70.

Various programs for controlling an operation of the OCT device, initial values, and the like may be stored in the memory 72. For example, a hard disk drive, a flash ROM, a USB memory detachably mounted on the OCT device, and the like can be used as the memory 72. Further, in the memory 72, various information about imaging may be stored in addition to the OCT image generated from the OCT data. The monitor 80 may display OCT data (OCT image).

### OCT Optical System

Next, the OCT optical system 100 in the present example will be described with reference to FIG. 2.

The OCT light source 11 of the present embodiment emits low coherent and broadband light. For example, the OCT light source 11 may be a super luminescent diode (SLD) light source.

A light splitter 15 splits light emitted from the OCT light source 11 into measurement light and reference light. FIG. 1 shows the light splitter 15 as a fiber coupler. As shown in FIG. 1, the measurement light is illuminated onto the subject eye through the light guiding optical system 40. Further, return light of the subject eye returns to the light guiding optical system 40 and is guided into the spectroscopic optical system 20. The reference light is guided into the spectroscopic optical system 20 through the reference optical system 30. In FIG. 1, the return light and the reference light of the measurement light are combined by a coupler (for example, the light splitter 15 in FIG. 1) and then guided into the spectroscopic optical system 20.

As shown in FIG. 1, the light guiding optical system 40 may include an optical scanner 41, an objective optical system 45, and the like. The optical scanner 41 is used to scan the measurement light on tissue of the subject eye. The measurement light scanned by the optical scanner 41 is scanned on the tissue of the subject eye through the objective optical system 45.

In a case of acquiring the fundus OCT, as shown in FIG. 1, the measurement light passing through the objective optical system 45 may be pivoted around one point (referred to as a pivot point). The pivot point is disposed in the anterior eye part through alignment, and thereby the fundus OCT is acquired.

The optical path length adjustment unit adjusts an optical path length difference between the measurement light and the reference light. In a case of capturing the fundus OCT, the optical path length difference is corrected in accordance with the individual difference in the axial length of each subject eye. Further, in a case where an anterior eye part OCT is captured, the optical path length difference is adjusted to a predetermined value. The optical path length adjustment unit changes an optical path length of at least one of the measurement optical path or the reference optical path. In FIG. 1, the optical path length is changed by moving an emission end of a fiber in the light guiding optical system 40 in the optical axis direction.

The spectroscopic optical system 20 of the present embodiment is used as a spectrometer. The spectroscopic optical system 20 detects a spectral interference signal between the reference light and the return light of the measurement light.

### Acquisition of Depth Information

The control unit 70 performs processing (Fourier analysis) on the spectral signal detected by the detector 120 to obtain OCT data of the subject eye. More specifically, the spectral interference signal may be rewritten as a function of wavelength λ and converted into a function I(k) at equal intervals with respect to the wave number k (=2π/λ). Further, Fourier transformed information may be represented as a signal including a real number component and an imaginary number component in a Z space. The control unit 70 may obtain the OCT data by obtaining an absolute values of the real number component and the imaginary number component of the signal in the Z space.

### Operation Explanation

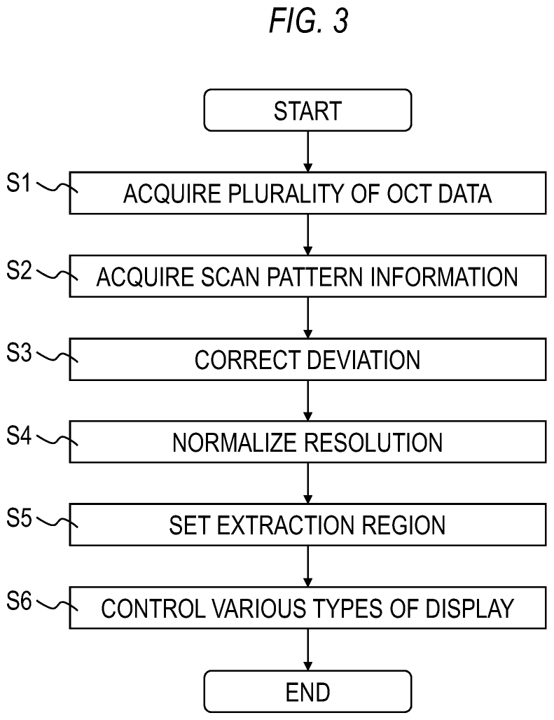

Next, an ophthalmologic image processing method executed by the OCT system of the example will be described with reference to the flowchart shown in FIG. 3. Each process of the flowchart may be executed by the control unit 70, on the basis of the ophthalmologic image processing program. In the present example, a plurality of OCT data acquired by a certain scan pattern are displayed on the monitor 80 by performing each processing of the flowchart. For convenience, the OCT data in the following description are all B scan data. The OCT data corresponding to the scan line is also referred to as a slice.

### S1: Acquisition Step

First, the OCT data of the subject eye is captured through various adjustments of the OCT optical system 100. The OCT data may be captured in any one of a plurality of predetermined scan patterns. First, a case where multi-scanning in one direction is performed will be described here. Further, unless otherwise specified, the OCT data in the present example will be described as an image being captured with a scan length of 9 mm.

The captured OCT data may be stored (saved) in the memory of the device in association with information indicating a scanning speed. Accordingly, the captured OCT data is acquired as a captured image by the control unit 70 (S1). In a case where the plurality of OCT data are captured at one time, the plurality of OCT data are acquired in the step of S1. In addition, information which indicates the scan pattern (scan pattern information) in a case where the plurality of OCT data is acquired, is acquired (S2). The scan pattern information may be associated with the plurality of OCT data. The scan pattern information may further include information which indicates a scan position in the individual OCT data.

### Deviation Correction

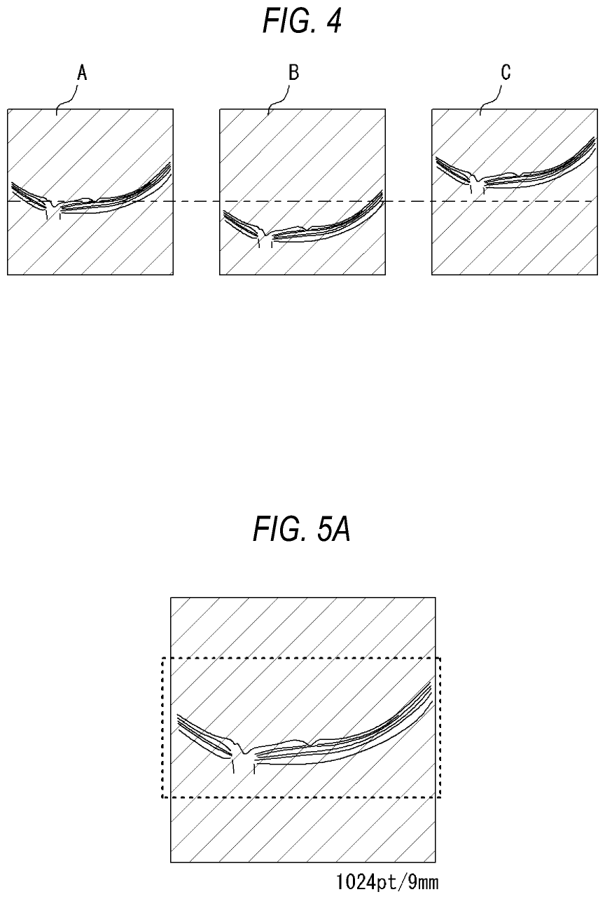

FIG. 4 shows an example of the plurality of OCT data acquired in the step of S1. It is assumed that the OCT data A, B, and C captured at different scan positions and by the multi-scanning in one direction. Since timings of capturing images of the plurality of OCT data are different from each other, the position of the image in each OCT data may be different, due to change in positional relationship between the device and the eye during imaging and change in the adjustment state of the eye.

On the other hand, in the present example, image processing for correcting the mutual positional relationship between the plurality of OCT data is performed (S3). In the present example, the other OCT data is corrected by affine transformation on the basis of any one of the plurality of OCT data such that the positions of at least one feature common to each OCT data match. As an example, in the present example, the mutual positional relationship of the plurality of OCT data is adjusted such that the positions of IS/OS of central fovea are matched.

### Resolution Normalization

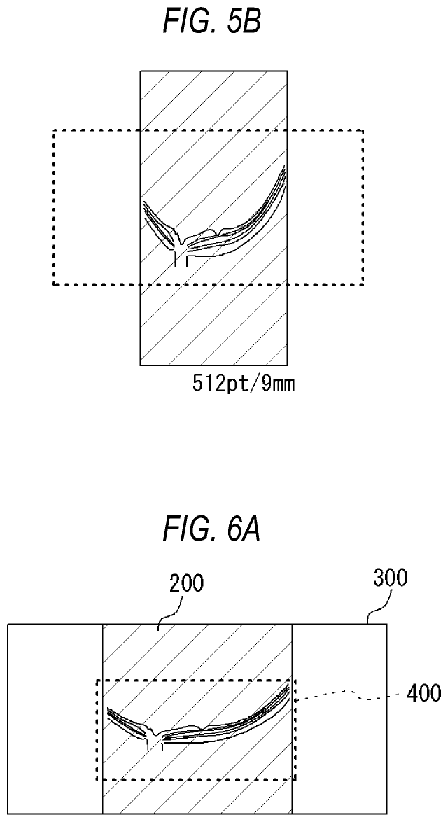

In the OCT data, the number of A scan OCT data (number of points) constituting the OCT data may differ from the scan length. In such a case, a display magnification in the transverse direction differs depending on the number of points (resolutions) with respect to the scan length. On the other hand, in the present example, a lateral magnification in the OCT data is normalized to be constant. Specifically, an A scan is interpolated (or thinned out) such that the number of points in the transverse direction in each OCT data becomes a constant value with respect to the scan length. For example, the A scan of the OCT data of 512 pt/9 mm shown in FIG. 5B is interpolated in accordance with the OCT data of 1024 pt/9 mm shown in FIG. 5A. As a result, the OCT data of 512 pt/9 mm shown in FIG. 5B is stretched twice in the transverse direction, and an image having substantially the same appearance as that of FIG. 5A is obtained. In the present example, since the number of points for the scan length in each OCT data is normalized, it is easy to observe the tissue of the subject eye without the user being aware of the A scan setting at the time of imaging.

### Extraction Region Setting

Next, the extraction region is set in the plurality of OCT data after the deviation correction is performed (S4). Here, FIG. 6A shows a relationship between the display region 300 and the OCT data 200 in a case where the entire OCT data 200 is displayed on the display region 300. As shown in FIG. 6A, in a case where the entire OCT data 200 is displayed, a region in which the image of the subject eye is not present may occupy most of the display region. Further, as shown in FIG. 6A, the image of the subject eye becomes relatively smaller due to the mismatch in aspect ratio between the image 200 and the display region 300.

On the other hand, in the present example, an extraction region 400 is set in a part of the OCT data 200, and an image region (extracted OCT data) included in the extraction region 400 is enlarged and displayed as shown in FIG. 6B. In such a case, a range which includes the image of the subject eye in the depth direction is detected in the OCT data 200. The detected range is the extraction region 400.

In the present example, the extraction region is set at the same position for the plurality of OCT data. Specifically, the extraction region is set to the same position for the plurality of OCT data after the positional deviation correction to include the image of the subject eye in each of the plurality of OCT data. In the present example, the extraction region is set in the plurality of OCT data such that the positional relationships of the extraction regions, with respect to the position of the IS/OS of the central fovea in the plurality of OCT data, match each other. In such a case, the extraction region may be set such that both an OCT data in which a position of the upper end of the image of the subject eye is at the shallowest position and an OCT data in which a position of the lower end of the image of the subject eye is at the deepest position, among the plurality of OCT data after the deviation correction, can be entirely viewed in the depth direction.

In the present example, the upper end and the lower end of the image of the subject eye are obtained on the basis of the detection result of a layer boundary with respect to the OCT data. The upper end of the extraction region is set on the basis of the upper end of the layer boundary on the shallowest layer side among the plurality of layer boundaries detected by the layer detection (segmentation processing). Likewise, the lower end of the extraction region is set on the basis of the lower end of the deepest layer boundary among the plurality of detected layer boundaries. In such a case, the upper end and the lower end of the extraction region may be set with a margin at a position separated by a certain distance from the upper end and the lower end at the layer boundary on the deepest layer side. In such a case, the margin on the upper end side may be provided such that the corpus vitreum is disposed in the extraction region. The margin on the lower end side may be provided such that the deep layer of the choroid layer is disposed in the extraction region.

The extracted OCT data is enlarged and displayed in accordance with an aspect ratio of the display region 300. The aspect ratio of the display region 300 is predetermined. In a case where the extracted OCT data (extraction region 400) is horizontally longer than the display region 300, a magnification for enlarged display of the extracted OCT data is obtained such that a width of the extracted OCT data matches a width of the display region 300. In a case where the extracted OCT data (extraction region 400) is vertically longer than the display region 300, the magnification for enlarged display of the extracted OCT data is obtained such that the widths of the extracted OCT data and the display region 300 in the vertical direction match each other.

### Display Control

In the display region 300 on the monitor 80, the plurality of extracted OCT data based on the multi-scanning are alternatively displayed (S6). The extracted OCT data displayed in the display region 300 is automatically or manually switched to the extracted OCT data captured at another scan position. In such a case, since the same position in the displayed plurality of OCT data is extracted, it is easy to appropriately grasp the difference in the tissue between the scan lines. Further, since the extracted OCT data corresponding to each scan position is set after considering the position of the image in the plurality of OCT data captured at each scan position in advance, the image of the subject eye is likely to be entirely viewed in the display region 300. Further, since the magnification in a case where the extracted OCT data corresponding to each scan position is displayed in the display region 300 is constant, the examiner is unlikely to feel uncomfortable before and after the extracted OCT data is switched to that at another scan position.

### Modification of Extraction Region

In the present example, the OCT data that is the source of the extracted OCT data remains even after the extracted OCT data is displayed. It may be possible to accept an operation of modifying the extraction region on the screen on which the extracted OCT data based on the initial extraction region is displayed. The position of the boundary in the extraction region (the position of the boundary in either the transverse direction or the depth direction) may be modified. On the basis of the operation, the extracted OCT data based on the modified extraction region is displayed. Thereby, the examiner is able to properly confirm the tissue even in a case where the initial extraction region is not set to the range intended by the examiner. For example, the setting position of the extraction region may be changed by scrolling the mouse wheel or operating a key on a keyboard.

### Display Setting for Each Scan Pattern

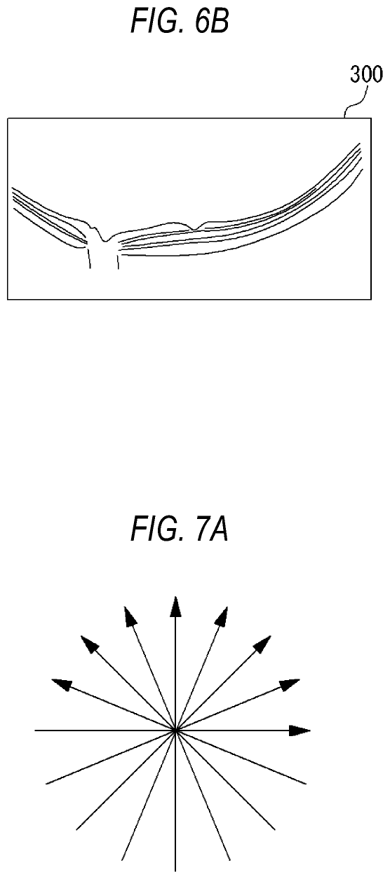

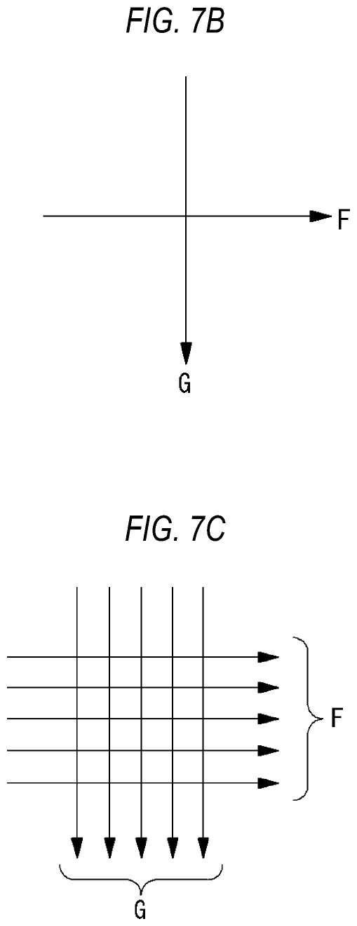

In the present example, it is predetermined for each type of the scan pattern whether the extraction region is set in the B scan OCT data captured at each scan position together with another B scan OCT data. For example, in a case of the above-mentioned one-way multi-scanning, the extraction region is set collectively for all OCT data. That is, the deviation correction and the setting of the extraction region are collectively performed for all the OCT data constituting the multi-scanning The same applies to a radial scanning shown in FIG. 7A. On the other hand, as shown in FIGS. 7B and 7C, in a cross scanning and a multi-cross scanning, the OCT data constituting the scan pattern is divided into groups for each scanning direction (divided into an F group and a G group in each of FIGS. 7B and 7C), and extraction regions are collectively set in each OCT data included in each group.

### MODIFICATION

Although the present disclosure has been described above on the basis of the embodiments and examples, the present disclosure is not necessarily limited to this, and various modifications are permitted.

For example, in the above-mentioned example, the scan length of the OCT data acquired by the device was fixed at 9 mm. On the other hand, in recent years, the angle of view has been widened, and there are devices with longer scan lengths. However, in observation and diagnosis using the fundus OCT, information about the central portion of the fundus is more important than information about the peripheral part.

Therefore, in a device having a long scan length (for example, 15 mm or more), a range of the extraction region in the transverse direction may be switchable between two predetermined values. For example, the range of the extraction region may be switchable between the range around the central fovea (for example, a range of about 9 mm) and the maximum range of the device. In such a case, along with the display region 300, GUI widgets corresponding to these two ranges may be provided on the screen. By operating the GUI widgets, the range of the extraction region in the transverse direction is switched. Whether the initial value of the extraction region is in the range around the central fovea centralis or the maximum range may be set in advance.

Further, in the above-mentioned example, the case where the extracted OCT data is displayed on the monitor 80 has been described, but the present invention is not limited to this. The extracted OCT data may be displayed in accordance with the display region of the report even in a case where the data is displayed in the report. The report may be an electronic media. The report may also be a paper medium.

For example, in the present embodiment described above, an ophthalmologic imaging device for capturing an image of OCT data of a subject eye has been described as an example, but the present invention is not limited thereto. For example, the present embodiment may be applied to a device for capturing an image of OCT data of an inspection target object. For example, the inspection target object may be a living body such as an eye, skin, or a blood vessel, or may be a sample other than a living body such as a resin body.

What is claimed is:

1. An ophthalmologic image processing method performed in an ophthalmologic image processing device, the ophthalmologic image processing method comprising:
an acquisition step of acquiring a plurality of B scan OCT data of a tissue of a subject eye, the plurality of B scan OCT data being captured at different scan positions;
a setting step of collectively setting an extraction region in the plurality of B scan OCT data to include an image of the subject eye in each of the plurality of B scan OCT data; and
a display control step of displaying, on a predetermined display region, a plurality of extracted OCT data extracted from the respective B scan OCT data, each of the plurality of extracted OCT data being switched to be alternatively displayed.

2. The ophthalmologic image processing method according to claim 1, further comprising:
a correction step of correcting a mutual positional relationship of the plurality of B scan OCT data,
wherein in the setting step, the extraction region is set in each corrected B scan OCT data after the mutual positional relationship is corrected.

3. The ophthalmologic image processing method according to claim 1,
wherein in the display control step, a lateral magnification of the extracted OCT data on the display region is adjusted, in accordance with a scan density of the B scan OCT data from which the extracted OCT data is extracted, to be constant among the B scan OCT data having different scan densities.

4. The ophthalmologic image processing method according to claim 1,
wherein the plurality of B scan OCT data acquired in the acquisition step are captured at different scan positions in an identical scan pattern.

5. The ophthalmologic image processing method according to claim 4,
wherein it is predetermined, for each type of the scan pattern, whether the extraction region is collectively set in the B scan OCT data captured at each scan position together with another B scan OCT data.

6. The ophthalmologic image processing method according to claim 1,
wherein in the setting step, a transverse direction range in which the extraction region is set in the plurality of B scan OCT data is selectable from a plurality of transverse direction ranges different from each other, and
the extraction region is collectively set in the selected transverse direction range of each of the plurality of B scan OCT data.

7. An ophthalmologic image processing device, comprising a processor configured to:
acquire a plurality of B scan OCT data of a tissue of a subject eye, the plurality of B scan OCT data being captured at different scan positions;
collectively set an extraction region in the plurality of B scan OCT data to include an image of the subject eye in each of the plurality of B scan OCT data; and
display, on a predetermined display region, a plurality of extracted OCT data extracted from the respective B scan OCT data, each of the plurality of extracted OCT data being switched to be alternatively displayed.

8. The ophthalmologic image processing device according to claim 7,
wherein the processor is configured to correct a mutual positional relationship of the plurality of B scan OCT data, and
when the processor collectively sets the extraction region, the extraction region is set in each corrected B scan OCT data after the mutual positional relationship is corrected.

9. The ophthalmologic image processing device according to claim 8,
wherein when the processor displays the plurality of extracted OCT data, a lateral magnification of the extracted OCT data on the display region is adjusted, in accordance with a scan density of the B scan OCT data from which the extracted OCT data is extracted, to be constant among the B scan OCT data having different scan densities.

10. The ophthalmologic image processing device according to claim 9,
wherein the acquired plurality of B scan OCT data are captured at different scan positions in an identical scan pattern.

11. The ophthalmologic image processing device according to claim 10,
wherein it is predetermined, for each type of the scan pattern, whether the extraction region is collectively set in the B scan OCT data captured at each scan position together with another B scan OCT data.

12. The ophthalmologic image processing device according to claim 11,
wherein when the processor collectively sets the extraction region, a transverse direction range in which the extraction region is set in the plurality of B scan OCT data is selectable from a plurality of transverse direction ranges different from each other, and
the extraction region is collectively set in the selected transverse direction range of each of the plurality of B scan OCT data.
